(19) United States
(12) Patent Application Publication
(10) Pub. No.: US 2008/0187982 A1
(43) Pub. Date: Aug. 7, 2008

(54) Methods for removal of non-living plant matter

(76) Inventors: Eric S. Heinz, Memphis, TN (US); John J. Krajewski, Wheeling, IL (US)

Correspondence Address: ERIC S. HEINZ, 3698 JOHNWOOD DR., MEMPHIS, TN, 38122, US

(21) Appl. No.: 11/670,974

(22) Filed: Feb. 3, 2007

(51) Int. Cl. C12N 1/21; C12N 1/14; C12N 1/20; C12N 1/15; C12N 1/10

(52) U.S. Cl. 435/252.3; 435/254.1; 435/252.1; 435/258.1; 435/254.11

(57) ABSTRACT

A method of accelerating decomposition of non-living plant material from a ground surface. The method employs the application of a saprotroph containing suspension to non-living plant material such as leaves or the stubble of a cultivated crop. The suspension is formulated with a pH and nutrient level whereby the physiologic activity of the saprotroph is optimized. The deposited saprotroph containing suspension accelerates the natural decomposition process without decomposing living plant material. A resulting humus is thereby formed which brings nutrients back into the ground surface.

### BACKGROUND

The invention deals with the field of cleaning up an area having a multiplicity of non-living plant matter dispersed thereon. The method employs a suspension containing a saprophyte which enables accelerated decomposition when applied to non-living plant matter.

Extensive areas of the world experience an annular cycle of climatic change. The flora of those regions does experience death and rebirth on a regular cycle. Various types of trees undergo color changes in the autumn because of chlorophyll variations as temperature and angle of incidence from the sun vary from the summer. While the changing colors offer beauty, there is a negative side. As autumn progresses, leaves fall from the trees to the ground.

A similar cycle of death and rebirth occurs in the farm fields of the world. Once crops such as corn, soybeans, cotton, and sugar cane are harvested, remnants of the crop, known in the industry as stubble, remain in the fields.

If left on the ground, leaves and stubble biologically degrade and deteriorate into a nutrient rich compound known as humus. While humus may fertilize the ground surface in preparation of spring growth, the process of its natural formation is slow, and decomposition of non-living plant matter tends to be messy and unsightly. For homeowners, the negative effects of dying and falling leaves becomes inconvenient. For farmers, the stubble also creates inconvenience in preparation of the fields for the next crop.

Current techniques for leaf removal are mechanical in origin, and involve raking or blowing of the leaves into a common pile. It becomes especially difficult when leaves fall into shrubs and flowerbeds. The leaf piles are then filled into plastic bags for pick-up and disposal. This process tends to be time-consuming and tiring. Physical injury such as back injuries and carpal tunnel syndrome are common in people who perform this work. Air pollution produced by leaf blowers, lawn vacuums, refuse collection trucks, and lawn mowers causes a significant environmental problem leading to increased health problems.

Farmers have to till down stubble in order to prepare the ground surface to accept seeds for a new crop. Tilling is also necessary to create irrigation pathways in the field. Multiple passes are often required, and as a result, pollution from farm equipment is generated, along with an increased expense for petroleum. In an effort to reduce pollution and fuel costs, some farmers are practicing a new “no-till” method of farming. These farmers allow the stubble to naturally decompose, however the ground surface is not optimal for seeding the next crop and as a result, harvest volumes can be reduced.

There exists a need for a new, more efficient method of non-living plant matter removal. This invention is directed to address the shortcomings of the mechanical means currently used to remove non-living plant matter. When employing the present method, much of the effort, possibility for injury, cost, and pollution can be obviated.

### SUMMARY

The present invention is a method for removing non-living plant matter that is present on a ground surface such as the lawn of a residential dwelling or a crop field. The method includes providing a suspension containing a saprotroph and spreading the suspension over the ground surface and the non-living matter thereon. Typically the suspension would consist of one or more species of saprotrophs, which are organisms that obtain their energy from non-living organic matter. The biological activity of saprotrophs found in nature, such as those extracted from natural sources such as soil or from the reticulorumen of ruminants such as cattle or sheep, is sufficient to digest non-living plant matter, however genetic engineering of these organisms may help enhance their effectiveness.

The pH of the solution may be modified in order to optimize the biological activity of the saprotroph. The physiologic processes of saprotrophs, and the effectiveness of the enzymes they produce, are sensitive to pH because changes in pH can alter the molecular structure of proteins. A conformational change in the shape of a protein making up an enzyme has the potential to cause deactivation of the enzyme. A conformational change of a protein found in the cell membrane of a saprotroph may lead to the death of the organism due to the inability of the protein to function transferring nutrients and wastes across the cell membrane. Modification of the suspension pH may be achieved with the use of acid/base buffers, which would be familiar to one in the field of biochemistry.

Nutrients may also be added to the solution in order to optimize the biological activity of the saprotroph. This may include urea, cellulose, sucrose, or glucose.

In a preferred embodiment, the suspension is applied to the non-living plant matter by means of a conventional spraying instrument. Tank Pump Sprayer, product number T-120, from The Liquid Fence Company located in Brodheadsville, Pa., is an example of a conventional spraying instrument. The majority of the surface area should be covered with the suspension to maximize the effectiveness of the method by increasing the surface area in contact with the suspension.

The suspension is allowed to dry, at which point the saprotroph would initiate it's decomposition of the plant matter by its release of cellulase and other enzymatic proteins. Saprotrophs decompose only non-living matter, so plants such as grass and perennials would not be affected by the suspension.

The purpose of this method is to accelerate the natural decomposition process. Once the non-living matter is decomposed, it is turned into a nutrient rich humus that fertilizes the ground surface.

The present invention is thus a method for conveniently, easily, and efficiently removing non-living plant matte from a ground surface. More specific features and advantages obtained in view of those features will become apparent with reference to the DETAILED DESCRIPTION OF THE INVENTION, appended claims, and accompanying drawing figure.

### BRIEF DESCRIPTION OF THE DRAWINGS

FIG. 1 is a view showing an individual applying a saprophyte containing suspension by spraying it in a liquid form to cover leaves on the ground.

### DETAILED DESCRIPTION

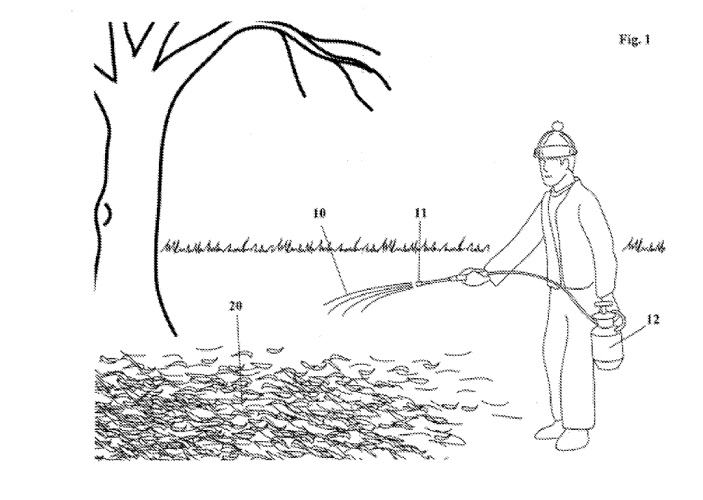

Referring now to the drawing figure, FIG. 1 illustrates an individual implementing the method in accordance with the present invention. The individual Is shown as carrying a canister 12 having a saprotroph containing suspension therewithin. The canister would, typically, be pressurized so as to channel the suspension 10 through a hose extending from the canister onto the leaves 20.

It will be understood that the intent of applying the suspension is to saturate the surfaces of the leaves with saprotroph organisms.

After application of the solution 10, the saprotrophs begin to decompose the leaves through biological means.

It will be understood that an appropriate saprotroph will be used to decompose non-living plant matter which that organism is most efficient at decomposing. For instance, tough leaves of a palm tree may be decomposed faster by organism A than organism B, which is efficient at decomposing oak tree leaves.

The present invention may be carried out in other specific ways than those herein set forth without departing from the scope and essential characteristics of the invention. For instance, this process may be used to accelerate the decomposition of non-living plant matter in wildfire prone regions, such as the Southern California. The present embodiments are, therefore, to be considered in all respects as illustrative and not restrictive, and all changes coming within the meaning and equivalency range of the appended claims are intended to be embraced therein.

1. A method of accelerating decomposition of non-living plant matter on a ground surface, comprising applying a genetically engineered saprotroph containing suspension to the non-living plant matter.

2. The method of claim 1 wherein the suspension has a pH between 3 and 8, dependent upon a given saprotroph, whereby the pH is of the value where the rate of decomposition is maximized.

3. The method in accordance with claim 1 wherein the suspension contains nutrients selected from the group consisting of urea, cellulose, sucrose, and glucose.

4. The method in accordance with claim 1 wherein the saprotroph is selected from the group consisting of fungi, bacterium, lichen, protozoan, and archaebacteria.

5. The method in accordance with claim 1 wherein the suspension contains a plurality of saprotroph species.

6. The method of claim 1 wherein the step of applying comprises spraying the suspension over non-living plant matter.

7. (canceled)

8. A method for removing a multiplicity of leaves on a ground surface, comprising the steps of:
providing a genetically engineered saprotroph containing suspension having a pH and nutrient level such that the physiologic activity of the saprotroph is optimized so that the saprotroph can decompose leaves at a rate faster than that of natural decomposition,
applying the suspension over the leaves by a conventional spraying instrument,
allowing the suspension to dry,
allowing the decomposition to form humus,
and allowing the humus to fertilize the ground surface.

9. (canceled)

10. The method of claim 8 wherein the physical state of the suspension is selected from the group consisting of the liquid state, the foam state, and the atomized state.

11. A method for removing stubble on a ground surface after cultivation of a crop, comprising the steps of:
providing a genetically engineered saprotroph containing suspension having a pH and nutrient level such that the physiologic activity of the saprotroph is optimized so that the saprotroph can decompose stubble at a rate faster than that of natural decomposition,
applying the suspension over the stubble by a conventional spraying instrument,
allowing the suspension to dry,
allowing the decomposition to form humus,
and allowing the humus to fertilize the ground surface.

12. (canceled)

13. The method of claim 11 wherein the physical state of the suspension is selected from the group consisting of the liquid state, the foam state, and the atomized state.
